(19) United States
(12) Patent Application Publication
(10) Pub. No.: US 2026/0088172 A1
(43) Pub. Date: Mar. 26, 2026

(54) OPHTHALMIC SYSTEM AND BEHAVIOR MODIFICATION SUPPORT METHOD

(71) Applicant: TOPCON CORPORATION, Tokyo (JP)

(72) Inventor: Noriyasu TAKEDA, Tokyo (JP)

(73) Assignee: TOPCON CORPORATION, Tokyo (JP)

(21) Appl. No.: 19/329,579

(22) Filed: Sep. 16, 2025

(30) Foreign Application Priority Data
Sep. 24, 2024 (JP) 2024-165293

(51) Int. Cl. G16H 50/20; A61B 3/00; A61B 3/18; A61B 5/00; A61B 5/16; G16H 10/60; G16H 20/70; G16H 40/20; G16H 40/67

(52) U.S. Cl. CPC G16H 50/20; A61B 3/0058; A61B 3/18; A61B 5/4842; A61B 5/7275; G16H 10/60; G16H 20/70; G16H 40/20; G16H 40/67; A61B 5/167

(57) ABSTRACT

An ophthalmic system includes a first information terminal owned by at least a patient and a second information terminal owned by a medical institution. The first information terminal includes at least a first processor configured to execute an application that manages information sharing and communication and a first data communication module configured to perform data communication with the second information terminal. The second information terminal includes at least a second processor configured to execute the application and a second data communication module configured to perform data communication with the first information terminal. The first processor is configured to set a specific function for prompting a behavior modification related to disease treatment of the patient in the application based on a result of a patient's personality diagnosis test executed by the application.

### CROSS-REFERENCE TO RELATED APPLICATION

This application is based on and claims priority under 35 USC 119 from Japanese Patent Application No. 2024-165293 filed on Sep. 24, 2024, the entire content of which is incorporated herein by reference.

### BACKGROUND

### 1. Field

The present disclosure relates to an ophthalmic system and a behavior modification support method.

### 2. Description of Related Art

In recent years, a hospital terminal such as a personal computer or a cloud computer and a patient terminal such as a personal computer, a tablet terminal, or a smartphone are connected to each other via a network, and a doctor can remotely diagnose the patient through data communication between the terminals. In addition, it is possible to check symptoms, receive consultations, and receive advice by using a function of a social networking system (SNS) between the patient terminal and the hospital terminal even outside of a hospital diagnosis time.

For example, JP6805581B discloses an information processing device that creates a conversation log by extracting important parts from text data indicating contents of a conversation between a doctor and a patient during a remote diagnosis. The information processing device acquires call information of a user from a terminal and creates the conversation log based on the call information. Further, the information processing device acquires a bookmark set in a conversational sentence included in the conversation log based on a user operation, and creates and outputs a bookmark log in which the bookmark is set based on the bookmark.

### SUMMARY OF INVENTION

Meanwhile, eye diseases such as glaucoma and age-related macular degeneration often require long-term outpatient visits, and it is known that the progression of such diseases can be suppressed by continuing to take medication or administer eye drops. These eye diseases are relatively susceptible to elderly people. Therefore, there is a problem that there is a certain number of patients who are unable to, for example, visit the hospital or continue taking medication or administering eye drops. In other words, it is difficult to continue taking medication or administering eye drops, which poses the problem of eye diseases gradually progressing.

The present disclosure has been made in view of such circumstances, and an object of the present disclosure is to provide an ophthalmic system and a behavior modification support method that maintain continuity of treatment for a disease by a patient and effectively support an improvement in motivation for the treatment.

An ophthalmic system includes: a first information terminal owned by at least a patient; and a second information terminal owned by a medical institution. The first information terminal includes at least a first processor configured to execute an application that manages information sharing and communication, and a first data communication module configured to perform data communication with the second information terminal. The second information terminal includes at least a second processor configured to execute the application, and a second data communication module configured to perform data communication with the first information terminal. The first processor is configured to set a specific function for prompting a behavior modification related to disease treatment of the patient in the application based on a result of a patient's personality diagnosis test executed by the application.

According to the present disclosure, it is possible to maintain continuity of treatment for a disease by a patient and effectively support an improvement in motivation for the treatment.

### BRIEF DESCRIPTION OF DRAWINGS

FIG. 1 is an overall view of an ophthalmic system according to the present illustrative embodiment;

FIG. 2 is a block diagram showing an example of a hardware configuration of a terminal device and a patient terminal according to the present illustrative embodiment;

FIG. 3 is a diagram showing a display example of a behavior modification image corresponding to type A;

FIG. 4 is a diagram showing a display example of a behavior modification image corresponding to type B;

FIG. 5 is a diagram showing a display example of a behavior modification image corresponding to type C;

FIG. 6 is a diagram showing a display example of a behavior modification image corresponding to type D; and

FIG. 7 is a flowchart showing an example of an operation procedure of a behavior modification support method according to the present illustrative embodiment in chronological order.

### DESCRIPTION OF EMBODIMENTS

Hereinafter, an example of the present disclosure will be described with reference to the drawings, but the scope of the present disclosure is not limited to the illustrative embodiment described here, and various modifications can be made without departing from the scope of the present disclosure.

### 1. Configuration of Ophthalmic System

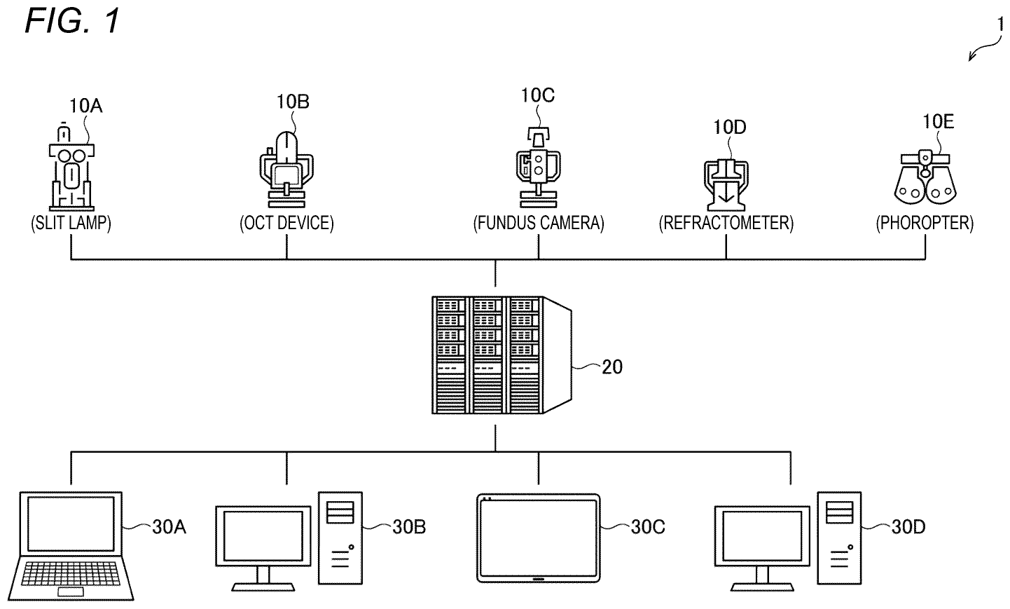

First, a configuration example of an ophthalmic system 1 according to the present illustrative embodiment will be described with reference to FIG. 1.

FIG. 1 is an overall view of the ophthalmic system 1 according to the present illustrative embodiment. The ophthalmic system 1 includes a plurality of ophthalmic devices, a data management computer 20, and a plurality of terminal devices 30A, 30B, 30C, and 30D. The data management computer 20 and each of the ophthalmic devices or communication devices connected to the ophthalmic devices, and the data management computer 20 and each of the terminal devices 30A to 30D are connected via a network. For example, an examination result obtained by an ophthalmic device is stored in the data management computer 20 via the network, so that a doctor or its assistant using a terminal device (for example, the terminal device 30A) can check the examination result. The number of ophthalmic devices and the number of terminal devices are merely examples, and are not limited.

As shown in FIG. 1, the ophthalmic devices include a slit lamp 10A, an OCT device 10B, a fundus camera 10C, a refractometer 10D, and a phoropter 10E. Each of the ophthalmic devices may be owned by different medical institutions, such as a general hospital, an ophthalmic clinic, and a university hospital, or a specific medical institution may collectively own a plurality of ophthalmic devices.

For example, the fundus camera 10C is a device owned by many medical institutions, and thus models with different manufacturers and functions are introduced. However, in the ophthalmic system 1, regardless of the manufacturer, the model, or the like, the doctor or its assistant can check the examination result, including information obtained by other medical institutions, on at least one of the terminal devices 30A to 30D.

Here, features of each ophthalmic device will be briefly described.

The slit lamp 10A, also known as a slit lamp microscope, is a device that obtains an anterior segment image, which is an image of a cross section of a cornea, by irradiating a subject eye, which is an eyeball of a subject, with elongated slit-shaped illumination light from an oblique direction. The slit lamp 10A is used to examine, for example, various anterior segments such as corneal shapes, corneal endothelial cells, and Meibomian glands.

The OCT device 10B is a device that obtains a tomographic image of a fundus (retina) by dividing low-coherence light into two parts, irradiating the fundus with one part, causing backscattered light of the one part to interfere with the other part, and receiving the light with a light receiving element such as a CCD image sensor. The tomographic image is used to determine eye diseases such as glaucoma, diabetic retinopathy, and retinal pigment degeneration. As the OCT device 10B, a device of any type such as a Fourier domain type, a time domain type, or a swept source type may be used.

The fundus camera 10C is a device that irradiates a fundus portion of the subject eye with illumination light and receives fundus reflected light by a light receiving element such as charge coupled devices (CCD) image sensor to acquire data of a fundus image. The fundus image is used to determine eye diseases such as glaucoma, diabetic retinopathy, and age-related macular degeneration.

The refractometer 10D is an examination device capable of measuring various eye characteristics such as an intraocular pressure value, an eye refractive power, and a corneal curvature of the subject eye.

The phoropter 10E, also known as vision tester, is an examination device used to select a lens suitable for the subject eye. The phoropter 10E includes a plurality of lenses, and by inserting one or a plurality of the lenses on the optical axis, the eye refractive power such as myopia, hyperopia, and astigmatism, or a binocular visual function can be measured.

The ophthalmic device 10 may include devices other than those described above. For example, the ophthalmic device may include a scanning laser ophthalmoscope which performs scanning using a light beam to irradiate the fundus and receives reflected light or fluorescence at a predetermined portion with a light receiving element such as a CCD to acquire image data of the fundus image. Another example is a specular microscope capable of imaging the corneal endothelial cells, measuring a density, and measuring the corneal thickness.

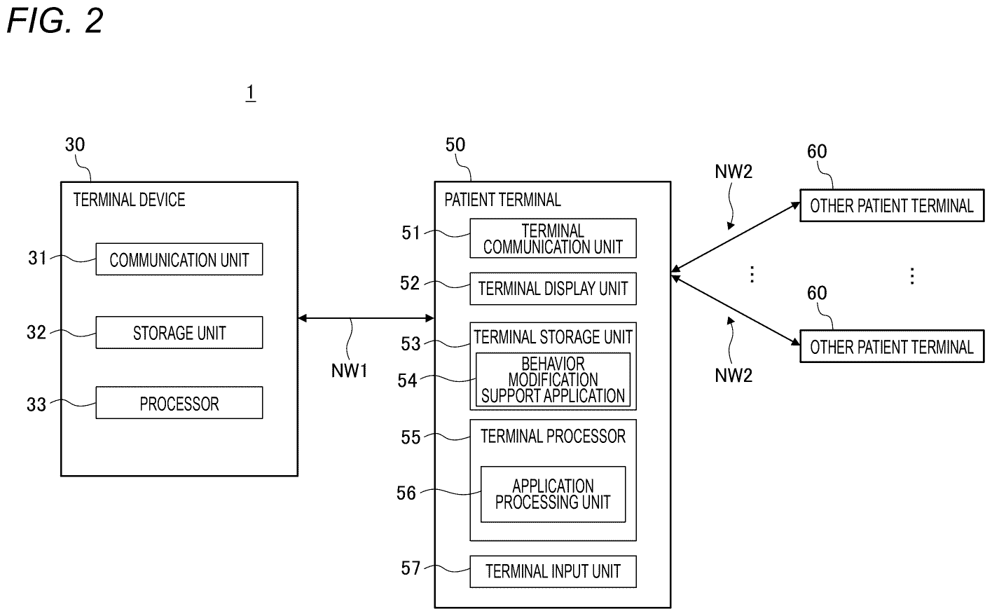

As will be described later with reference to FIG. 2, the terminal devices 30A to 30D each include an internal storage unit 32, which stores data such as the examination result, the image data, and the like of the ophthalmic device (see above) transmitted via the network. In addition, each of the terminal devices 30A to 30D is connected to the data management computer 20, so that it is possible to access data such as the examination result stored in the data management computer 20 at any time. A type of the data management computer 20 is not particularly limited, and for example, the data management computer 20 may be configured as a cloud type server or may be configured as an on-premise server disposed in a facility such as a medical institution.

Each of the terminal devices 30A to 30D is a computer such as a PC, a notebook PC, a tablet terminal, or the like provided in each medical institution. Software for using the ophthalmic system 1 may be installed in each of the terminal devices 30A to 30D. Therefore, a doctor or its assistant can check the examination result and the image data stored in the data management computer 20 on each of the terminal devices 30A to 30D. Further, since a doctor or its assistant can confirm recorded items of an electronic medical record in addition to data such as the examination results acquired by other medical institutions, information such as the presence or absence of a disease of the subject eye is determined in consideration of each piece of information. Although not illustrated in FIG. 1, the ophthalmic system 1 according to the present illustrative embodiment may further include a patient terminal (see FIG. 2) owned by each patient who visits a medical institution such as a hospital for treatment (disease treatment) to suppress an eye disease.

Next, an example of the hardware configuration of the terminal device 30, a patient terminal 50, and other patient terminals 60 constituting the ophthalmic system 1 according to the present illustrative embodiment will be described with reference to FIG. 2. FIG. 2 is a block diagram showing the example of the hardware configuration of the terminal device 30 and the patient terminal 50 according to the present illustrative embodiment. The terminal device 30 in FIG. 2 corresponds to each of the terminal devices 30A to 30D in FIG. 1. Although an example of the hardware configuration of the other patient terminal 60 is not illustrated in FIG. 2, the other patient terminal 60 has the same configuration as the patient terminal 50. In the following description, an example will be described as a use case in which the ophthalmic system 1 according to the present illustrative embodiment maintains and improves the motivation of a patient who visits the ophthalmology due to some kind of eye disease to continue treatment for the disease.

The terminal device 30, which is mainly used by a doctor, and the patient terminal 50 are connected to each other via a network NW1 so as to be able to perform data communication with each other. Similarly, the patient terminal 50 and the other patient terminal 60 are connected via a network NW2 so as to be able to perform data communication with each other. Each of the networks NW1 and NW2 is a so-called wireless network, and may be a communication form using, for example, a wireless LAN such as Wi-Fi (registered trademark) that is available in the facility, or short-range wireless communication such as Bluetooth (registered trademark). Although not shown in FIG. 2, the terminal device 30 and the other patient terminal 60 may be connected via the network NW1 so as to be able to perform data communication with each other.

The terminal device 30 includes a communication unit 31, a storage unit 32, and a processor 33. The terminal device 30 is configured as a computer such as a PC located inside a medical institution (in a hospital).

The communication unit 31 is connected to each type of ophthalmic device (see FIG. 1) disposed inside the medical institution or the patient terminal 50 via a network (not shown), and includes a communication circuit that performs data communication with each type of ophthalmic device or the patient terminal 50. The communication unit 31 performs data communication with each type of ophthalmic device or the patient terminal 50, receives various data transmitted from each type of ophthalmic device or the patient terminal 50, and transmits various data to each type of ophthalmic device or the patient terminal 50.

The storage unit 32 includes, for example, a random access memory (RAM) and a read only memory (ROM), and temporarily stores a program necessary for the operation of the terminal device 30 and data acquired or generated during the operation. The RAM is, for example, a work memory used during the operation of the terminal device 30. The ROM stores and holds, for example, a program for controlling the terminal device 30 in advance. The storage unit 32 stores a program and data of a behavior modification support application, which will be described in detail later. The behavior modification support application is executed by the processor 33, and data communication is performed between the terminal device 30 and the patient terminal 50 during the execution of the behavior modification support application.

The processor 33 includes, for example, at least one of a central processing unit (CPU), a digital signal processor (DSP), a graphical processing unit (GPU), or a field programmable gate array (FPGA). The processor 33 is an example of a second processor, and functions as a controller that controls the overall operation of the terminal device 30. The processor 33 performs a control process for controlling the operation of each unit of the terminal device 30, a data input and output process between each unit of the terminal device 30, a data calculation process, and a data storage process. The processor 33 operates according to a program stored in the storage unit 32. The processor 33 uses the storage unit 32 during operation, and temporarily stores data generated or acquired by the processor 33 in the storage unit 32.

The patient terminal 50 includes a terminal communication unit 51, a terminal display unit 52, a terminal storage unit 53, a terminal processor 55, and a terminal input unit 57. The patient terminal 50 is a computer owned or carried by a patient who visits a hospital for treatment of a disease such as an eye disease, and is implemented by, for example, a notebook PC, a tablet terminal, or a smartphone. The patient terminal 50 can install the behavior modification support application in an executable manner upon approval by a patient who visits a medical institution (for example, a hospital).

The terminal communication unit 51 is connected to the terminal device 30 or the other patient terminal 60 via a network (not shown), and is configured to use a communication circuit that performs data communication with the terminal device 30 or the other patient terminal 60. The terminal communication unit 51 performs data communication with the terminal device 30 or the other patient terminal 60, receives various data transmitted from the terminal device 30 or the other patient terminal 60, and transmits various data to the terminal device 30 or the other patient terminal 60.

The terminal display unit 52 displays results (for example, a display screen) based on various processes performed by the terminal processor 55. The terminal display unit 52 is, for example, a display such as a liquid crystal display (LCD) or an organic EL. The terminal display unit 52 and the terminal input unit 57 that receives an operation from a patient, who is a main user of the patient terminal 50, may be integrally configured, and in this case, the terminal input unit 57 and the terminal display unit 52 are implemented by a touch panel display.

The terminal storage unit 53 includes, for example, RAM and ROM, and temporarily stores a program necessary for the operation of the patient terminal 50 and data acquired or generated during the operation. The RAM is, for example, a work memory used during the operation of the patient terminal 50. The ROM stores and holds, for example, a program for controlling the patient terminal 50 in advance. The terminal storage unit 53 stores a program and data of a behavior modification support application 54, which will be described in detail later. The behavior modification support application 54 is executed by the terminal processor 55, and data communication is performed between the terminal device 30 and the patient terminal 50 during the execution of the behavior modification support application 54.

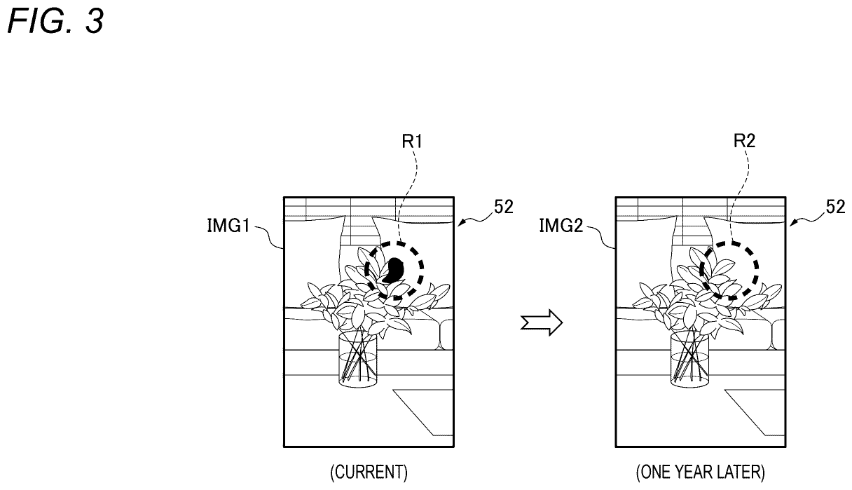

The behavior modification support application 54 may be installed in an executable manner in each of the terminal device 30, the patient terminal 50, and the other patient terminal 60. The behavior modification support application 54 may be processed and executed by the patient terminal 50 alone, or may be processed and executed by data communication with another device (for example, the terminal device 30 or the other patient terminal 60) via a network (for example, the network NW1 or NW2). The behavior modification support application 54 executes at least a function of performing a personality diagnosis test on a patient and a specific function (to be described later) of prompting behavior modification related to disease treatment of a patient who visits a medical institution (for example, a hospital) based on a result of the personality diagnosis test. Details of the specific function of the behavior modification support application 54 will be described later with reference to FIGS. 3 to 6.

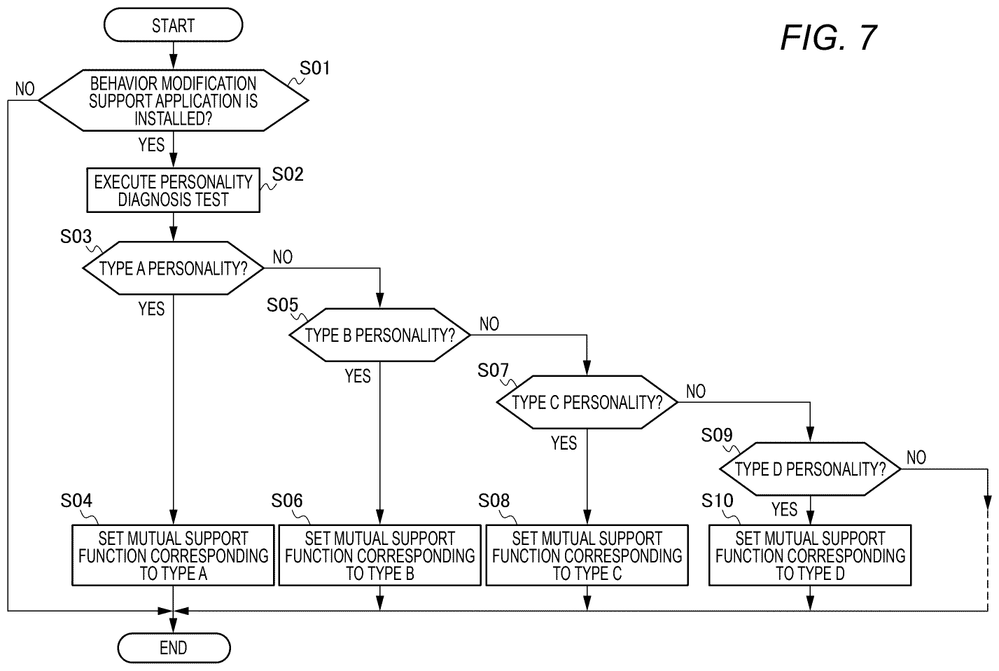

The terminal processor 55 is an example of a first processor, and includes at least one of CPU, DSP, GPU, and FPGA, for example. The terminal processor 55 functions as a controller that controls the overall operation of the patient terminal 50. The terminal processor 55 performs a control process for controlling the operation of each unit of the patient terminal 50, a data input and output process between each unit of the patient terminal 50, a data calculation process, and a data storage process. The terminal processor 55 operates according to a program stored in the terminal storage unit 53. The terminal processor 55 uses the terminal storage unit 53 during operation, and temporarily stores data generated or acquired by the terminal processor 55 in the terminal storage unit 53. In the patient terminal 50, the terminal processor 55 is configured to execute various processes (for example, the processes in the flowchart shown in FIG. 7) in cooperation with the terminal storage unit 53. The terminal processor 55 cooperates with the terminal storage unit 53 to achieve the functions of an application processing unit 56.

The application processing unit 56 starts, executes, and ends the behavior modification support application 54 stored in the terminal storage unit 53, and displays a processing result being executed (for example, a personality diagnosis test screen, screens illustrated in each of FIGS. 3 to 6) on the terminal display unit 52. Further, the application processing unit 56 detects an operation (for example, an input operation of an answer to a question in a personality diagnosis test) on the terminal input unit 57 by the patient who owns the patient terminal 50, generates a notification screen based on the detection result, and displays the notification screen on the terminal display unit 52. In addition, the application processing unit 56 generates the notification screen (for example, see FIGS. 3 to 6) according to a result of the personality diagnosis test (that is, the personality of the patient), receives and acquires the notification screen from the terminal device 30, and displays the result on the terminal display unit 52.

The terminal input unit 57 includes a device that detects an operation by the patient who owns the patient terminal 50, and corresponds to, for example, at least one of a touch panel, a touch pad, a button, a microphone, and a mouse. The terminal input unit 57 detects an operation by the patient while the behavior modification support application 54 is being executed by the application processing unit 56, and sends the detection result to the application processing unit 56.

The other patient terminal 60 is owned by another patient different from the patient who owns the patient terminal 50. Specifically, the other patient terminal 60 is a computer owned or carried by a patient who, like the patient who owns the patient terminal 50, visits a hospital for treatment of a disease such as an eye disease, and is implemented by, for example, a notebook PC, a tablet terminal, or a smartphone. The other patient terminal 60 can install the behavior modification support application in an executable manner upon approval by another patient who visits a medical institution (for example, a hospital). Although a detailed hardware configuration example of the other patient terminal 60 is not illustrated in FIG. 2, it is the same as the hardware configuration example of the patient terminal 50.

### 2. Notification Screens Displayed According to Personality

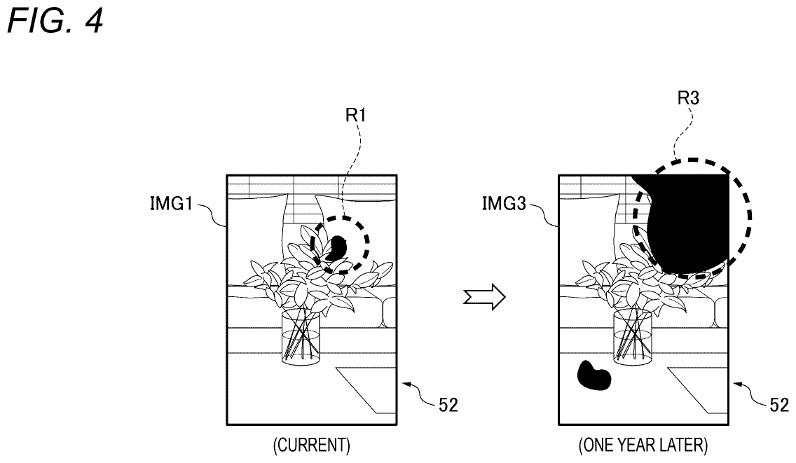

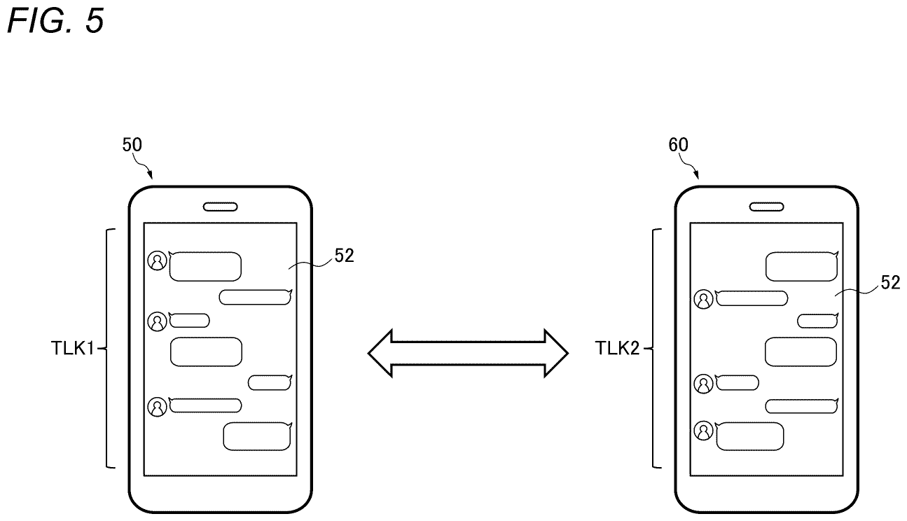

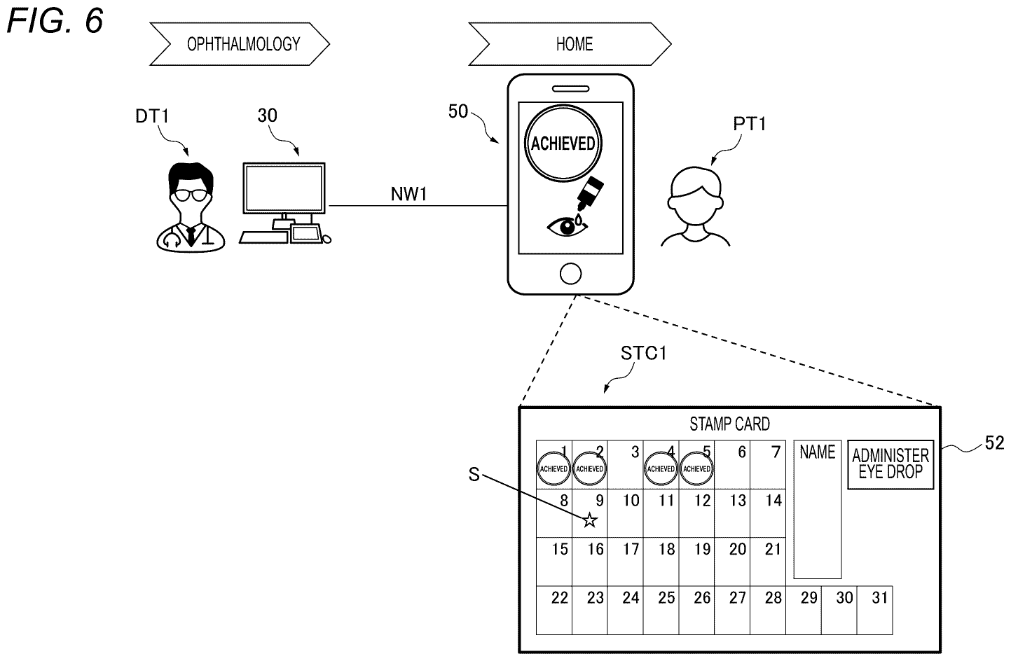

Next, examples of the notification screen displayed by the behavior modification support application 54 according to the personality of a patient will be described with reference to FIGS. 3 to 6. FIG. 3 is a diagram showing a display example of a behavior modification image corresponding to type A. FIG. 4 is a diagram showing a display example of a behavior modification image corresponding to type B. FIG. 5 is a diagram showing a display example of a behavior modification image corresponding to type C. FIG. 6 is a diagram showing a display example of a behavior modification image corresponding to type D.

FIG. 3 illustrates an example in which the behavior modification support application 54 displays a current image IMG1 (an example of a first image) and a future image IMG2 (an example of a second image) on the terminal display unit 52 of the patient terminal 50 in a comparative manner for a patient diagnosed to have a personality of type A (for example, positive). The current image IMG1 is an example of a first image, and shows a visual field image as it appears under a current disease state of the patient. On the other hand, the future image IMG2 is an example of a second image, and indicates a future visual field image that is expected to be seen in a case where the medication (for example, eye drops) is continuously taken (that is, treated) for a certain period of time (for example, one year) with reference to the present. That is, the current image IMG1 shows a state in which a defect is present in a part (region R1) of the field of view, while the future image IMG2 shows a state in which the defect in the field of view is reduced (region R2) as a result of a patient having a positive personality continuing to take medication and a somewhat normal field of view is obtained. This is expected to effectively enhance the patient's motivation to adhere to treatment using a praise-and-encouragement approach.

FIG. 4 illustrates an example in which the behavior modification support application 54 displays a current image IMG1 (an example of a first image) and a future image IMG3 (an example of a second image) on the terminal display unit 52 of the patient terminal 50 in a comparative manner for a patient diagnosed to have a personality of type B (for example, acting in response to a sense of crisis). The current image IMG1 is the same as that of FIG. 3. The future image IMG3 is an example of a second image, and indicates a future visual field image that is expected to be seen in a case where the medication (for example, eye drops) is not continuously taken (that is, treated) for a certain period of time (for example, one year) with reference to the present. That is, the current image IMG1 shows a state in which a defect is present in a part (region R1) of the field of view, while the future image IMG3 shows a state in which the defect in the field of view is further deteriorated as a result of a patient having a personality that is prone to acting in response to a sense of crisis not continuing to take medication, leading to constriction in the field of view (region R3, region R4), which interferes with the daily life. This is expected to effectively improve the motivation of the patient for treatment through a strict management policy.

FIG. 5 illustrates an example in which the behavior modification support application 54 displays, on the terminal display unit 52 of the patient terminal 50, a communication screen (for example, a talk screen TLK1) between a patient diagnosed as having a personality of type C (for example, extroverted) and another patient who owns the other patient terminal 60. Similarly, an example is shown in which the behavior modification support application 54 displays, on the terminal display unit 62 of the other patient terminal 60, a communication screen (for example, a talk screen TLK2) between another patient diagnosed as having a personality of type C (for example, extroverted) and a patient who owns the patient terminal 50.

When installing the behavior modification support application 54, the patient terminal 50 detects an approval operation by the patient to share information and communicate with other patient terminals 60, and then installs the behavior modification support application 54. Similarly, when installing the behavior modification support application 54, the other patient terminal 60 also detects an approval operation by another patient to share information and communicate with the patient terminal 50, and then installs the behavior modification support application 54. As a result, for the patient and other patients with extroverted personalities, the patient and other patients suffering from similar diseases can share information and communicate (for example, talk screens TLK1 and TLK2) using the behavior modification support application 54, exchanging information that can be made public, thereby helping each other to change the mood and feel more positive about disease treatment. The information that may be made public includes, but is not limited to, information such as gender, age, hospital name, treatment period, disease name, and next scheduled hospital visit date.

FIG. 6 illustrates an example in which the behavior modification support application 54 displays a medication history table STC1 on the terminal display unit 52 of the patient terminal 50 for a patient PT1 diagnosed as having a personality of type D (for example, conscientious), based on the detection of an input operation indicating that the patient PT1 has taken (for example, applied) a medication (for example, eye drops) provided by a medical institution (for example, a hospital or a pharmacy). In response to an operation input every time the patient PT1 takes the medication (for example, eye drops), the behavior modification support application 54 updates the display so as to put a stamp on the medication history table STC1 to indicate that eye drops have been administered. The medication history table STC1 can be viewed by the terminal device 30 that can perform data communication with the patient terminal 50. As a result, a doctor DT1 in the medical institution (for example, a hospital) can appropriately monitor whether the patient PT1 is administering eye drops on a daily basis. In addition, since the patient PT1, who has a conscientious personality, can visually and explicitly check the past records of administering eye drops in the medication history table STC1 using the behavior modification support application 54, the motivation for disease treatment can be appropriately maintained and improved.

A next hospital visit date may be displayed in the medication history table STC1 (star mark S in the drawing). In addition, it may be possible to check the medication history table STC1 for the next month onwards, and there may be a function of showing a scheduled end date of treatment or hospital visit. Such a display also leads to maintaining and improving the motivation of the patient PT1 for the disease treatment.

Although not illustrated, for a patient diagnosed as having a personality that easily gets bored, the behavior modification support application 54 frequently displays new information on a disease the patient is suffering from or other information on the terminal display unit 52, and also displays a comment from a doctor sent from the terminal device 30 on the terminal display unit 52. As a result, the patient having a personality that easily gets bored can adequately maintain and improve motivation for disease treatment by frequently receiving new information.

Although not illustrated, for a patient diagnosed as having a profit-driven personality, the behavior modification support application 54 performs a process of providing an incentive (for example, benefits or points) for taking (for example, applying) medication (for example, eye drops) in association with the identification information of the patient. As a result, the patient who has a profit-driven personality can receive an incentive (for example, benefits or points) by taking (for example, applying) the medication (for example, eye drops), and thus it is possible to appropriately maintain and improve motivation for disease treatment.

### 3. Operation Procedure of Ophthalmic System (Behavior Modification Support Method)

Next, an example of an operation procedure of the ophthalmic system 1 according to the present illustrative embodiment will be described with reference to FIG. 7. FIG. 7 is a flowchart showing an example of the operation procedure of the behavior modification support method according to the present illustrative embodiment in chronological order. The series of processes shown in FIG. 7 may be executed mainly by the terminal processor 55 of the patient terminal 50 alone, or may be executed in cooperation with the terminal device 30 via data communication.

In FIG. 7, the terminal processor 55 determines whether the behavior modification support application 54 is installed (S01). When it is determined that the behavior modification support application 54 is not installed (S01, NO), the process of FIG. 7 by the terminal processor 55 ends.

On the other hand, when it is determined that the behavior modification support application 54 is installed (S01, YES), the terminal processor 55 executes a personality diagnosis test in the behavior modification support application 54 to diagnose the personality of a patient who owns the patient terminal 50 (S02). Here, it is preferable that the personality diagnosis test includes three to five questions and can be completed in a few minutes. Accordingly, the behavior modification support application 54 can execute a specific function (see FIGS. 3 to 6) for continuing the disease treatment of the patient according to the personality of the patient.

The terminal processor 55 determines whether the result of the personality diagnosis test executed in step S02 is a type A personality (S03). In a case where it is determined that the patient has a type A personality (that is, positive personality) (S03, YES), the terminal processor 55 performs a process of setting mutual support function corresponding to type A (S04). Specifically, the terminal processor 55 displays the current image IMG1 showing a visual field image corresponding to a current disease situation of the patient and the future image IMG2 showing a visual field image expected if the patient continues taking the medication for a certain period of time in a comparative manner (see FIG. 3). As a result, the patient having the positive personality can appropriately recognize the current self and the future self if he or she does not neglect continuing the treatment, and thus can maintain and improve motivation for the disease treatment.

On the other hand, in a case where it is determined that the patient does not have the type A personality (that is, positive personality) (S03, NO), the terminal processor 55 determines whether the result of the personality diagnosis test executed in step S02 is a type B personality (S05). In a case where it is determined that the patient has a type B personality (that is, personality that acts in response to a sense of crisis) (S05, YES), the terminal processor 55 performs a process of setting mutual support function corresponding to type B (S06). Specifically, the terminal processor 55 displays the current image IMG1 showing a visual field image corresponding to a current disease situation of the patient and the future image IMG3 showing a visual field image expected if the patient does not continue taking the medication for a certain period of time in a comparative manner (see FIG. 4). As a result, the patient having the personality that acts in response to a sense of crisis can appropriately recognize the current self and the future self if the he or she neglects continuing the treatment, and thus can maintain and improve motivation for the disease treatment.

On the other hand, in a case where it is determined that the patient does not have the type B personality (that is, personality that acts in response to a sense of crisis) (S05, NO), the terminal processor 55 determines whether the result of the personality diagnosis test executed in step S02 is a type C personality (S07). In a case where it is determined that the patient has a type C personality (that is, extroverted personality) (S07, YES), the terminal processor 55 performs a process of setting mutual support function corresponding to type C (S08). Specifically, the terminal processor 55 displays the communication screen (for example, a talk screen TLK1) between the patient and another patient who owns the other patient terminal 60 in the behavior modification support application 54, and updates the display of the content of the conversation every time a conversation with the other patient terminal 60 takes place (see FIG. 5). As a result, a patient having an extroverted personality can share useful information with other patients undergoing treatment for the same disease, and can easily engage in conversation and write about their experiences, and thus can maintain and improve motivation for the disease treatment.

On the other hand, in a case where it is determined that the patient does not have the type C personality (that is, extroverted personality) (S07, NO), the terminal processor 55 determines whether the result of the personality diagnosis test executed in step S02 is a type D personality (S09). In a case where it is determined that the patient has a type D personality (that is, conscientious personality) (S09, YES), the terminal processor 55 performs a process of setting mutual support function corresponding to type D (S10). Specifically, in the behavior modification support application 54, the terminal processor 55 displays the medication history table STC1 and updates the display content thereof based on the detection of the input operation indicating that the patient PT1 has taken the medication provided by the medical institution (see FIG. 6). As a result, since the patient, who has a conscientious personality, can visually and explicitly check the past records of administering eye drops in the medication history table STC1, the motivation for disease treatment can be appropriately maintained and improved.

Although not illustrated in FIG. 7, for a patient diagnosed as having a personality type different from types A to D, the terminal processor 55 similarly sets (configures) a mutual support function corresponding to the personality of the patient in the behavior modification support application 54. As a result, the patient can receive an appropriate suggestion that motivates him or her to undergo the disease treatment from the behavior modification support application 54 according to the diagnosed personality of the patient, and thus it is possible to appropriately maintain and improve motivation for disease treatment.

The present disclosure is not limited to the illustrative embodiment described above and may be implemented in various modes without departing from the gist thereof. For example, the personality diagnosed by the behavior modification support application 54 is not necessarily limited to one, and in a case where a plurality of personalities are diagnosed, mutual support functions suited to each personality may be combined, set, and executed.

The image content used in the current image and the future image described with reference to FIGS. 3 and 4 may be adaptively selected according to the attribute (for example, occupation) of the patient. This selection is executed by, for example, the terminal device 30. For example, in a case where the occupation of the patient is a carrier who performs work such as track transportation, the current image and the future image may be an image of traffic lights as seen from a vehicle, or an image of an object that would be problematic if the patient had a visual field defect.

The ophthalmic system and the behavior modification support method according to the present disclosure provide the operations and effects listed below.

(1) An ophthalmic system including:
a first information terminal owned by at least a patient; and
a second information terminal owned by a medical institution,
wherein the first information terminal includes at least a first processor configured to execute an application that manages information sharing and communication, and a first data communication module configured to perform data communication with the second information terminal,
wherein the second information terminal includes at least a second processor configured to execute the application, and a second data communication module configured to perform data communication with the first information terminal, and
wherein the first processor is configured to set a specific function for prompting a behavior modification related to disease treatment of the patient in the application based on a result of a patient's personality diagnosis test executed by the application.

In the ophthalmic system according to the present disclosure, the application is executed while data communication is performed between the first information terminal and the second information terminal. The first information terminal owned by the patient sets, in the application, the specific function for prompting a behavior modification related to the disease treatment of the patient according to the result of the patient's personality diagnosis test executed by the application, and executes the specific function. As a result, the ophthalmic system can maintain continuity of the treatment for the disease of the patient and effectively support the improvement of the motivation for the treatment.

(2) In the ophthalmic system according to the present disclosure, the first information terminal further includes a first display, the second processor is configured to select a first image showing a current image of a patient's disease and a second image showing a predictive image of the disease treatment based on a status of the disease treatment of the patient, and transmit the selected first image and second image to the first information terminal, and the first processor is configured, as part of the specific function, to display the first and second images which are selected by the second processor on the first display.

The second information terminal selects the current image (first image) of the disease of the patient and the future prediction image (second image) for the disease treatment according to the status of the disease treatment of the patient, and shares the images with the first information terminal. The first information terminal displays, as the specific function, the first image and the second image selected by the second information terminal on the first display in the application. As a result, the ophthalmic system can visually and comparatively present the first image and the second image to the patient according to the result of the personality diagnosis test of the patient, and can effectively prompt the patient to maintain and improve the motivation to continue the treatment.

(3) In the ophthalmic system according to the present disclosure, the second processor is configured to reselect the first and second images based on a progress in the patient's disease treatment, and transmit the reselected first and second images to the first information terminal, and the first processor is configured, as part of the specific function, to display the reselected first and second images on the first display.

The second information terminal reselects the first image and the second image according to a progress degree of the status of the disease treatment of the patient, and shares the images with the first information terminal. This reselection may be repeated as appropriate depending on a period in which the disease treatment is necessary. As a result, the ophthalmic system adaptively selects the first image and the second image according to the progress degree of the status (progress status) of the disease treatment of the patient, and effectively prompts the patient to maintain and improve the motivation to continue the treatment.

(4) In the ophthalmic system according to the present disclosure, the first information terminal further includes an input interface, and the first processor is configured, as part of the specific function, to display a medication history table of a medication for the patient on the first display unit, and update the medication history table in response to an operation on the input interface by the patient who has taken the medication.

The first information terminal displays the medication history table of the patient on the first display, and updates the content of the medication history table in response to the operation on the input interface by the patient who has taken the medication. As a result, the ophthalmic system can appropriately update the medication history table by having a patient with a conscientious personality perform a predetermined operation on the input interface every time he or she takes the medication, and can effectively prompt the patient to maintain and improve the motivation to continue the treatment according to the personality of the patient.

(5) In the ophthalmic system according to the present disclosure, the medication history table includes at least one of a next hospital visit date and a scheduled end date of the disease treatment.

The medication history table includes at least one of the next hospital visit date and the scheduled end date of the disease treatment of the patient. The next hospital visit date is a date on which the patient is scheduled to visit the hospital next time. The scheduled end date of the disease treatment cannot be said to be the exact date on which the disease treatment ends, but is a date on which the disease treatment is expected to end depending on the progress of the disease. As a result, the ophthalmic system updates the content of the medication history table that includes at least one of the next hospital visit date and the scheduled end date of the disease treatment of the patient, so that it is possible to effectively prompt a patient having the conscientious personality to maintain and improve the motivation to continue the treatment.

(6) In the ophthalmic system according to the present disclosure, a third information terminal owned by a different patient and configured to perform data communication with the second information terminal and to use the application is further provided, and the first processor is configured to execute, as the specific function, the information sharing and the communication using the application with the third information terminal, when a first approval operation by the patient to approve the different patient and a second approval operation by the different patient to approve the patient are detected.

The third information terminal owned by the different patient can perform data communication with the second information terminal, and can use the application in the same manner. If there is mutual recognition between the patient who owns the first information terminal and the different patient who owns the third information terminal, the information sharing and communication using the application can be performed between the first information terminal and the third information terminal. As a result, for patients with an extroverted personality, the ophthalmic system can effectively prompt the patient to maintain and improve the motivation for the disease treatment by enabling them to share necessary information and communicate with other patients suffering from similar diseases (for example, eye diseases) and increasing their network of acquaintances.

(7) In the ophthalmic system according to the present disclosure, the second image is an image showing a predicted post-treatment field of view of the disease, if the patient continues treatment of the disease.

An image showing the predicted post-treatment field of view of the disease is displayed on the first information terminal if the patient continues treatment of the disease. As a result, for patients having a forward (positive) personality, the ophthalmic system visually and explicitly shows a bright future state if they continue the disease treatment, so that it is possible to effectively prompt the patient to maintain and improve the motivation for the disease treatment.

(8) In the ophthalmic system according to the present disclosure, the second image is an image showing a predicted post-deterioration field of view of the disease if the patient does not continue the treatment of the disease.

An image showing the predicted post-deterioration field of view of the disease is displayed on the first information terminal if the patient does not continue the treatment of the disease. As a result, for patients having a personality that only acts in response to a sense of crisis, the ophthalmic system visually and explicitly shows an anxious future state that gets worse if they do not continue the treatment of the disease, so that it is possible to effectively prompt the patient to maintain and improve the motivation for the disease treatment.

(9) An behavior modification support method executed by an ophthalmic system including a first information terminal owned by at least a patient, and a second information terminal owned by a medical institution, the behavior modification support method including:
executing, by the first information terminal, an application that manages information sharing and communication, and performing data communication with the second information terminal;
executing, by the second information terminal, the application and performing data communication with the first information terminal; and
setting, by the first information terminal, a specific function for prompting a behavior modification related to disease treatment of the patient in the application based on a result of a patient's personality diagnosis test executed by the application.

In the behavior modification support method, the application is executed while data communication is performed between the first information terminal and the second information terminal. The first information terminal owned by the patient sets, in the application, the specific function for prompting a behavior modification related to the disease treatment of the patient according to the result of the personality diagnosis test for the patient executed in the application, and executes the specific function. As a result, the ophthalmic system can maintain continuity of the treatment for the disease of the patient and effectively support the improvement of the motivation for the treatment.

What is claimed is:

1. An ophthalmic system comprising:
a first information terminal owned by at least a patient, the first information terminal including at least:
a first processor configured to execute an application that manages information sharing and communication; and
a first data communication module configured to perform data communication with the second information terminal; and
a second information terminal owned by a medical institution, the second information terminal including at least:
a second processor configured to execute the application; and
a second data communication module configured to perform data communication with the first information terminal,
wherein the first processor is configured to set a specific function for prompting a behavior modification related to disease treatment of the patient in the application based on a result of a patient's personality diagnosis test executed by the application.

2. The ophthalmic system according to claim 1,
wherein the first information terminal further includes a first display,
wherein the second processor is configured to select a first image showing a current image of a patient's disease and a second image showing a predictive image of the disease treatment based on a status of the disease treatment of the patient, and transmit the selected first and second images to the first information terminal, and
wherein the first processor is configured, as part of the specific function, to display the first image and the second image which are selected by the second processor on the first display.

3. The ophthalmic system according to claim 2,
wherein the second processor is configured to reselect the first and second images based on a progress in the patient's disease treatment, and transmit the reselected first and second images to the first information terminal, and
wherein the first processor is configured, as part of the specific function, to display the reselected first and second images on the first display.

4. The ophthalmic system according to claim 2,
wherein the first information terminal further includes an input interface, and
wherein the first processor is configured, as part of the specific function, to display a medication history table of a medication for the patient on the first display, and update the medication history table in response to an operation on the input interface by the patient who has taken the medication.

5. The ophthalmic system according to claim 4, wherein the medication history table includes at least one of a next hospital visit date and a scheduled end date of the disease treatment.

6. The ophthalmic system according to claim 2, further comprising:
a third information terminal owned by a different patient and configured to perform data communication with the second information terminal and to use the application,
wherein the first processor is configured to execute, as the specific function, the information sharing and the communication using the application with the third information terminal, when a first approval operation by the patient to approve the different patient and a second approval operation by the different patient to approve the patient are detected.

7. The ophthalmic system according to claim 2, wherein the second image is an image showing a predicted post-treatment field of view of the disease, if the patient continues treatment of the disease.

8. The ophthalmic system according to claim 2, wherein the second image is an image showing a predicted post-deterioration field of view of the disease if the patient does not continue the treatment of the disease.

9. A behavior modification support method executed by an ophthalmic system including a first information terminal owned by at least a patient, and a second information terminal owned by a medical institution, the behavior modification support method comprising:
executing, by the first information terminal, an application that manages information sharing and communication, and performing data communication with the second information terminal;
executing, by the second information terminal, the application and performing data communication with the first information terminal; and
setting, by the first information terminal, a specific function for prompting a behavior modification related to disease treatment of the patient in the application based on a result of a patient's personality diagnosis test executed by the application.
